(19) United States
(12) Patent Application Publication
(10) Pub. No.: US 2013/0211318 A1
(43) Pub. Date: Aug. 15, 2013

(54) WOUND THERAPY DEVICE

(71) Applicant: PAUL HARTMANN AG, (US)

(72) Inventors: Pierre Croizat, Herbrechtingen (DE); Axel Eckstein, Heidenheim (DE)

(73) Assignee: PAUL HARTMANN AG, Heidenheim (DE)

(21) Appl. No.: 13/761,801

(22) Filed: Feb. 7, 2013

Related U.S. Application Data
(60) Provisional application No. 61/598,474, filed on Feb. 14, 2012.

(30) Foreign Application Priority Data
Feb. 11, 2012 (EP) EP 12000901.4

(51) Int. Cl. A61M 3/02; A61M 35/00

(52) U.S. Cl. CPC A61M 3/0283; A61M 35/00
USPC 604/23; 604/305; 604/290

(57) ABSTRACT

Wound therapy device for the treatment of wounds by means of negative pressure and fluid irrigation, includes a negative-pressure dressing, a pressure sensor which can generate pressure measurement values in order to determine the negative pressure present in the negative-pressure dressing, a drainage container for collecting liquid, and a drainage line by means of which liquid can be conveyed from the negative-pressure dressing into the drainage container. The device can further include a first pump unit for aspirating liquid from the wound space via the drainage line, an instillation container for making available an instillation fluid, an instillation line. Instillation fluid can be delivered from the instillation container into the wound space.

### CROSS REFERENCE TO RELATED APPLICATIONS

The present application claims priority to U.S. Provisional Application Ser. No. 61/598,474 filed Feb. 14, 2012, which is herein incorporated by reference in its entirety.

### BACKGROUND OF THE ART

The present invention relates to a device for use in the negative-pressure therapy of wounds, in particular a device for the treatment of wounds by means of negative pressure and fluid irrigation. The invention further relates to a method for metering a suitable volume of instillation fluid for instillation therapy, and a method for determining a suitable volume of instillation fluid for instillation therapy of wounds.

The term negative-pressure therapy is understood here as meaning a method for the treatment of wounds in which negative pressure is applied in the wound space and liquid is aspirated from a wound. A wound space is understood as the space formed between a fluid-tight cover film and the wound base (body tissue in the wound area).

The expression “negative pressure” designates an air pressure in the wound space lower than the ambient air pressure (atmospheric air pressure). In connection with the invention, the pressure difference between the air pressure inside the wound dressing and the ambient air pressure is given in mmHg (millimeter of mercury), since this is customary in the field of negative-pressure therapy. 1 mmHg corresponds to one ton or 133.322 Pa (Pascal). In connection with the invention, the negative pressure, i.e. the pressure difference between the air pressure in the wound space and the ambient air pressure, is given as a positive numerical value in mmHg. A rise in the negative pressure consequently signifies an increase in the pressure difference between the air pressure in the wound space and the ambient air pressure. A drop in the negative pressure consequently signifies a decrease in the pressure difference between the air pressure in the wound space and the ambient air pressure.

Devices for the negative-pressure therapy of wounds, and wound dressings as a component part of such devices, are known in the prior art. Thus, for example, WO1993/009727 describes a device for promoting the wound-healing process by applying negative pressure to the skin region presenting the wound and surrounding the wound. The device according to WO1993/009727 comprises a vacuum means for generating the negative pressure, an airtight covering of the wound, which covering is referred to as a sealing means and is operatively connected to the vacuum means, and also a wound dressing referred to as a screening means and intended to be positioned on the wound inside the sealing means. The screening means is a porous polymer foam, for example a polyester foam. According to the description of WO1993/009727, the healing of different types of wounds, for example burns or pressure sores, can be accelerated by using negative-pressure therapy.

The negative-pressure therapy of wounds with simultaneous or alternate irrigation of the wound with a fluid (instillation fluid) is also referred to as instillation therapy. In the context of the present patent application, the term instillation fluid is used as a generic term for instillation liquids and instillation gases.

In the prior art, negative-pressure therapy systems for the treatment of wounds are known in which irrigation of the wound with an instillation fluid is possible. Such a device is also referred to as an instillation therapy device. By way of example, reference is made in this connection to WO2001/037922, which describes an instillation therapy device for the treatment of wounds by means of negative pressure and fluid irrigation.

Frequently used instillation liquids are water, saline solutions, in particular isotonic saline solutions, or medical solutions such as Ringer's solution. The instillation liquids can contain active substances, e.g., antiseptic substances, antibiotics, growth factors and clotting factors, or analgesics.

Frequently used instillation gases are air, oxygen-enriched air, or nitrogen.

An important advantage of instillation therapy over conventional negative-pressure therapy is that the wound can be irrigated with an instillation fluid without changing the dressing and without a long period of interruption of the negative-pressure therapy. If appropriate, the instillation fluid can remain in the wound for a defined time and is then aspirated by means of negative pressure. Typical time periods are from a few minutes to a few hours. During the period the instillation fluid remains in the wound, said instillation fluid can detach necrotic tissue, for example, and cell debris. In this way, the instillation therapy can contribute to debridement of the wound. Moreover, the instillation fluid can promote a wound environment that is favorable to the wound-healing process, for example by influencing the pH value of the wound or by improving the supply of oxygen. A wound environment in the acid pH range in particular can promote the wound-healing process. Optionally, the instillation fluid can also comprise active substances that support the wound-healing process.

In the instillation therapy of wounds, the problem of metering the instillation fluid always arises. If instillation fluid is delivered in too small a volume, the effect sought in conjunction with the fluid irrigation may not be achieved. Moreover, too small a volume of instillation fluid can have the effect that active substances contained in the instillation fluid, e.g. antibiotics or growth factors, are present in too small a quantity in the wound. The desired effect of promoting the wound-healing process can then only be achieved inadequately, if at all. If instillation fluid is delivered in too large a volume, this can lead to an overpressure inside the wound dressing. An overpressure of this kind generated in the wound space can damage the wound and lead to maceration of the intact skin surrounding the wound. In addition, an overpressure can lead to overstretching or distension of the dressing, which impairs the fit of the dressing on the wound. An overpressure in the wound space can also lead to a loss of leaktightness of the dressing, such that the negative-pressure dressing has to be changed before schedule. An additional change of the wound dressing can damage the healing wound and cause the patient pain. A change of dressing, which often has to be done in the operating theater in the case of serious wounds, also takes up a considerable amount of the medical personnel's time and is associated with added costs.

The quantity of instillation fluid can be controlled, for example, by introducing the instillation fluid and visually monitoring the bulging of the wound dressing. When the wound dressing starts to bulge, this shows the user of an instillation therapy device that enough instillation fluid has been introduced. The delivery of further instillation fluid must then be interrupted. A disadvantage of this method of metering the required quantity of instillation fluid is that the suitable time for interrupting further delivery of instillation fluid is estimated subjectively by the user and may therefore differ from case to case.

WO2010/033272A1 describes a device that is suitable for irrigating a wound with an irrigation liquid. According to the treatment method described in WO2010/033272A1, the irrigation liquid flows through the wound and is conveyed, together with wound exudate, into a container. The irrigation liquid is preferably hypertonic and causes body fluid to flow into the wound by osmosis. The volume of the liquid recovered from the wound can be established using a measuring device on the container, in order to monitor the therapy. The quantity of recovered wound liquid is derived from the difference between the volume of the irrigation liquid introduced into the wound and the measured volume.

WO2009/071924A1 discloses a method for determining the volume of a wound, which involves measuring the quantity of air that has to be aspirated from the wound to generate a defined negative pressure. The determination of the air quantity makes it possible to calculate or estimate the wound volume. The wound volume thus determined serves for metering a suitable volume of instillation fluid. A disadvantage of this method is that the wound volume can only be determined indirectly via the quantity of air removed from the wound. Since air is a compressible gas, the measured quantity of air can fluctuate according to the negative pressure at the same wound volume. Moreover, the calculation of the wound volume depends on changing parameters, such as the composition of the air, the air humidity, and the air temperature.

The problem addressed by the present invention is therefore to make available an improved method for instillation therapy of wounds that avoids the stated disadvantages of the prior art. In particular, the metering and the determination of a suitable volume of instillation fluid are to be improved. Moreover, the metering and determination of a suitable volume of instillation fluid are to be simplified and automated.

### SUMMARY OF THE INVENTION

As a solution to this problem, the invention proposes a wound therapy device for the treatment of wounds by means of negative pressure and fluid irrigation, in accordance with the device described herein. The device according to the invention thus comprises a negative-pressure dressing with a fluid-tight cover, such that a wound space can be formed between the cover and the wound base, a pressure sensor means, which can generate pressure measurement values, in fluidic communication with the wound space, a drainage container for collecting liquid, and a drainage line by means of which liquid can be conveyed from the wound space into the drainage container. The device further comprises a first pump unit for aspirating liquid from the wound space via the drainage line, wherein the first pump unit is connected in a fluid-conducting manner to the drainage container. The device further comprises an instillation container for making available an instillation fluid, an instillation line by means of which instillation fluid can be delivered from the instillation container into the wound space, a means for delivering an instillation fluid from the instillation container into the wound space, and a control means, wherein the control means can receive pressure measurement values from the pressure sensor means. According to the invention, the device is characterized in that, when a predefined upper negative-pressure threshold value is reached in the wound space, the control means can output a signal to switch off the first pump unit, and, when a predefined lower negative-pressure threshold value is reached in the wound space, the control means can output a signal to interrupt the delivery of the instillation fluid. An “upper negative-pressure threshold value” is here understood as a threshold value in the negative-pressure range which is customarily used for negative-pressure therapy, for example 100 mmHg or 125 mmHg. By contrast, a “lower negative-pressure threshold value” is understood as a threshold value which, compared to the “upper negative-pressure threshold value”, lies nearer the ambient air pressure, for example 0 mmHg or 25 mmHg.

A negative-pressure dressing within the meaning of the invention is a dressing which is suitable for the negative-pressure therapy of wounds and which is able to close off a wound space in a gastight manner and permits fluidic communication with a negative-pressure source and with an instillation fluid source. The negative-pressure dressing comprises a fluid-tight cover. In the simplest case, the drainage and/or instillation line can be routed under the edge of the fluid-tight cover, or through the cover, into the wound space. At least one means for the attachment of a drainage and/or instillation line is preferably present. In particular, the at least one means for the attachment of a drainage and/or instillation line is a negative-pressure attachment piece (port). Suitable negative-pressure attachment pieces are described, for example, in the patent applications WO2011/076340 and WO2011/091952 or in the unpublished patent application DE102011108726.9 of the applicant Paul Hartmann AG. A negative-pressure attachment piece that permits simultaneous attachment of drainage line and instillation line is disclosed in the unpublished patent application DE102011082341.7 of the applicant Paul Hartmann AG.

The fluid-tight cover is usually a self-adhesive cover film. In connection with the invention, it is possible, for example, to use the finished product Hydrofilm® (Paul Hartmann AG, Heidenheim, Germany) as suitable cover film. Moreover, the negative-pressure dressing usually comprises a wound pad, for example a textile wound pad or a foam, in particular an open-cell polymer foam. The wound pad can be designed with a single layer or multiple layers. For example, the commercial products VivanoMed® Foam, TenderWet® or ES-Compress from Paul Hartmann AG (Heidenheim, Germany) can be used individually or in combination as wound pad. The wound pad can have an additional wound contact layer, for example an ointment-impregnated wound pad, in particular an ointment-impregnated wound pad with an antimicrobial action. The ointment-impregnated wound pad can preferably be a silver-containing polyamide knit. A silver-containing polyamide knit is commercially available under the name Atrauman Ag® from the manufacturer Paul Hartmann AG, Heidenheim, Germany. Other commercial products suitable for an additional wound contact layer are the wound pads Branolind®, Grassolind® or Hydrotüll® from Paul Hartmann AG.

The pressure sensor means is in fluidic communication with the wound space and can generate pressure measurement values. The pressure sensor means can be in the form of one or more pressure sensors, in particular miniaturized electronic pressure sensors. If more than one pressure sensor is provided, the sensors can, if appropriate, be present at different positions on the wound therapy device. As pressure sensor, it is possible in principle to use any sensor that is suitable for determining the pressure of a fluid. The pressure sensor means must be configured such that pressure measurement values can be generated and forwarded via a communication link.

A communication link within the meaning of the invention can be any kind of suitable electrical signal transfer device, for example a power cable, a USB cable, a cable for serial data transfer, or a cable for parallel data transfer. The communication link can also be a radio link, for example a W-LAN link or a Bluetooth link. Moreover, the communication link can also be based on optical signals, for example on light signals, which are transferred in a fiber optic cable. The communication link can likewise comprise interfaces for information transfer with infrared light or a machine-to-machine link.

The device comprises a drainage container for collecting liquid that is recovered from the wound or from the wound space by means of the negative pressure. The liquid collected in the drainage container can be wound liquid and also instillation liquid located in the wound space. The container is preferably a vessel that is impermeable to liquid and gas and that does not substantially deform under the pressure conditions prevailing in negative-pressure therapy and instillation therapy, for example a canister made of plastic. The drainage container is preferably a disposable article that is discarded after use.

The device comprises a drainage line, by means of which liquid can be conveyed from the wound space into the drainage container. In the context of the present invention, a line, for example a drainage line, an instillation line or an irrigation line, is understood as any kind of fluid-conducting connection. A line is preferably a hose that is impermeable to liquid and gas. Such a hose is preferably made of plastic, in particular of silicone. Flat and flexible hoses have proven particularly useful in therapy, since flat lines cause less pain when the patient lies on them.

The negative pressure in the wound space is generated by a first pump unit. A pump unit comprises a conventional pump suitable for medical applications. The pump unit preferably comprises a diaphragm pump, e.g. diaphragm pumps from the manufacturer KNF (Freiburg, Germany) or Thomas (Puchheim, Germany). The pump model 1420VP-6VDL from the Thomas company is particularly suitable. An important advantage of a diaphragm pump is that, with this design form, no parts of the pump come into contact with the fluid. In this way, contamination of the pump unit by microorganisms is greatly reduced, the pump unit is made easier to clean and its useful life is extended. The use of a peristaltic pump would also be conceivable and advantageous. The negative pressure present in the wound space causes aspiration of fluid (wound exudate and possibly instillation fluid) from the wound. The fluid passes through the drainage line into the drainage container. For this purpose, the first pump unit is connected to the drainage container in a fluid-conducting manner. Preferably, the drainage container is arranged in the fluid path between the first pump unit and the drainage line. The first pump unit is in this case situated downstream of the drainage container, such that the pump unit provides the negative pressure to the wound dressing via the drainage container and through the drainage line. A protective filter or protective valve, which automatically closes when the container is full, is as far as possible mounted here between drainage container and pump unit so as to reliably prevent contamination of the pump with wound liquid. At the end toward the wound, the drainage line is secured on the negative-pressure dressing preferably by means of a negative-pressure attachment piece (port). In addition to securing the line, the port permits the production of a fluidic communication between drainage line and wound space.

The control means can control the first pump unit, i.e. it can switch the first pump unit on and off and control the stroke rate of the pump. The control means controls the first pump unit by means of control signals in particular. Moreover, the control means can receive pressure measurement values from the pressure sensor means and, on the basis of the pressure measurement values, determine the negative pressure in the wound space. This permits permanent monitoring of the negative pressure in the wound space by the control means. Correspondingly, the course of the negative-pressure therapy can be precisely controlled by the control means. In particular, when a predefined upper negative-pressure threshold value is reached in the wound space, the control means can output a signal to switch off the first pump unit, in order to avoid generating too high a negative pressure in the wound. Too high a negative pressure can damage the wound and impede the wound-healing process. The user of the wound therapy device is preferably informed, by suitable display means, of the applied negative pressure and/or of the time profile of the negative-pressure therapy. Using suitable input means such as buttons, a keyboard, a touchscreen or a computer interface, it is possible to input complex programs for time-controlled negative-pressure therapy. It is particularly advantageous for the wound-healing process if the negative-pressure therapy is carried out intermittently. With intermittent negative-pressure therapy, the level of the applied negative pressure is varied at regular intervals. For example, the negative pressure is lowered after a predetermined time period has elapsed. After another predetermined time period has elapsed, the negative pressure is raised again.

The wound therapy device according to the invention has an instillation container for making available an instillation fluid. The instillation container can be a canister, for example, or a bag impermeable to liquid. In this connection, it is possible in principle to use reusable containers or disposable containers. The instillation container is preferably a disposable container, in particular a disposable container that has been filled with a sterile instillation fluid at the time of manufacture. The instillation container can be connected to the wound space in a fluid-conducting manner via the instillation line. Moreover, the device according to the invention comprises a means for delivering an instillation fluid from the instillation container into the wound space. The means in question is preferably a second pump unit (fluid delivery pump) suitable for delivering liquids. The fluid delivery pump preferably comprises a diaphragm pump or a peristaltic pump.

The wound therapy device of the present invention is further characterized in that, when a predefined lower negative-pressure threshold value is reached in the wound space, the control means can output a signal to interrupt the delivery of the instillation fluid. An interruption in the delivery of instillation fluid avoids a situation where further introduction of instillation fluid in the wound generates a pressure that substantially exceeds the ambient pressure. A pressure above the ambient pressure could damage the wound and impair the wound-healing process. In the method proposed according to the invention, the interruption in the delivery of instillation fluid ensures at the same time that, in the instillation therapy, a suitable volume of instillation fluid is introduced into the wound.

After a suitable volume of instillation fluid has been introduced, the pressure in the wound space corresponds substantially to the ambient pressure. When using a wound pad composed of an open-cell polyurethane foam (for example the VivanoMed Foam® from Paul Hartmann AG), a substantial approximation of the negative pressure in the wound dressing to the ambient pressure generally takes place when the volume of the instillation fluid introduced into the wound amounts to two thirds of the volume of the wound space closed off by a cover film.

The present invention further comprises a method for metering a suitable volume of instillation fluid for the instillation therapy of wounds. The method for metering a suitable volume of instillation fluid is carried out by first making available a wound therapy device according to the invention. After applying the negative-pressure dressing to the wound and attaching drainage line and instillation line, the first pump unit is switched on in order to generate a negative pressure in the wound space. Any liquids present in the wound space are then aspirated through the drainage line into the drainage container. The pressure in the wound space is monitored by means of a pressure sensor means. When an upper negative-pressure threshold value is reached, for example 100 mmHg or 125 mmHg, the first pump unit is switched off. After the first pump unit has been switched off, instillation fluid from the instillation container is delivered through the instillation line into the wound space until a lower negative-pressure threshold value is reached, for example 10 mmHg or 20 mmHg. When a lower negative-pressure threshold value is reached, the further delivery of instillation fluid into the wound is ended particularly by the fact that the means for delivering the instillation fluid is inactivated and/or a valve arranged between instillation container and wound dressing interrupts the further delivery of instillation fluid.

The method according to the invention preferably comprises determining a suitable volume of instillation fluid for the instillation therapy of wounds, and storing the determined volume. If a suitable volume of instillation fluid is applied at the start of the instillation therapy (first irrigation cycle), it is advantageous if, for example, such a volume of instillation fluid or a previously defined fraction of the original volume is applied to the wound in subsequent irrigation cycles. To this end, it is proposed that the volume of instillation fluid metered according to the invention in the first irrigation cycle is determined and is stored, at least temporarily, for the metering of further irrigation cycles. A suitable volume of instillation fluid can be established, for example, by determining the duration of the first delivery of instillation fluid, i.e. the time span until the lower threshold value is reached. If the delivery rate of the means for delivering the instillation fluid is known, it is easy to calculate the volume of instillation fluid applied until interruption of the delivery of instillation fluid. In such cases, renewed determination of a suitable volume of instillation fluid is necessary only after a change of dressing.

According to a particularly preferred embodiment, the present invention comprises a wound therapy device which is suitable for carrying out the above-described method for metering a suitable volume of instillation fluid in the instillation therapy of wounds. In particular, the present invention comprises a wound therapy device which is suitable for carrying out the above-described method for determining a suitable volume of instillation fluid for instillation therapy and storing the determined volume.

The pressure sensor means can be arranged in the wound space, i.e. in the space formed between cover film and wound base. The pressure sensor means, for example in the form of one or more pressure sensors, can be integrated in a wound pad, for example in a wound pad composed of open-cell polyurethane foam. It has surprisingly been found that the negative pressure present in the wound space can also be determined very precisely if the pressure sensor means is provided at any desired part of the device, as long as the pressure sensor means is in fluidic communication with the wound space. The negative pressure in the wound space is determined on the basis of the pressure measurement values forwarded from the pressure sensor means to the control means. In order to improve the precision of the determination of the negative pressure in the wound space, additional processing of the pressure measurement values may be carried out if appropriate, for example using a correction algorithm.

The fluidic communication between the pressure sensor means and the wound space can be provided, for example, via the drainage line. For example, it would be possible for one or more pressure sensors to be arranged in the drainage line or in a line section branching off from the drainage line. It is equally possible that at least one pressure sensor is arranged in the drainage container or in a line section branching off from the drainage container. If the first pump unit is arranged downstream of the drainage container, it is possible in particular that the pressure sensor means is arranged in a line section arranged between drainage container and first pump or in a line section branching off from this line.

According to another preferred embodiment, the wound therapy device has a separate pressure measurement line, wherein the pressure measurement line is in fluidic communication with the wound space and with the pressure sensor means.

According to another particularly preferred embodiment, the wound therapy device has a ventilation line or an irrigation line, wherein the ventilation line or irrigation line is in fluidic communication with the wound space and with the pressure sensor means.

A ventilation line is understood here as an additional line which leads to the negative-pressure dressing and into the wound space and by means of which ambient air can be introduced into the wound space for the purpose of pressure equalization or for flushing through the fluid system. The ambient air delivered should be passed through a bacteria filter in order to rule out contamination of the wound space with germs. The bacteria filter can be present, for example, on the end of the ventilation or irrigation line directed away from the wound. The end of the ventilation or irrigation line directed toward the wound is in fluidic communication with the wound space. The end of the ventilation line or irrigation line directed away from the wound is in fluidic communication with the environment via a controllable ventilation valve or irrigation valve, such that ventilation or irrigation of the wound space and of the drainage line is permitted when the valve receives an opening impulse. The ventilation valve or irrigation valve is normally an electrically activated valve, preferably a magnetic valve which communicates with the control means. During the operation of the pump unit, the magnetic irrigation valve can be briefly opened, for example for 100 milliseconds, by the control means, for example at predetermined intervals. Since the ventilation or irrigation line is in fluidic communication with the drainage line via the wound space, air puffs are generated in the drainage line after a brief opening of the valve. These air puffs in the drainage line can loosen any agglomerations that are present and in this way can reduce the risk of the drainage line being occluded by wound liquid and tissue residues in negative-pressure mode. As long as the ventilation or irrigation valve is closed to the environment, a negative pressure corresponding substantially to the wound space can be measured in the interior of the ventilation or irrigation line. It is therefore possible to arrange pressure sensor means in the interior of such a ventilation or irrigation line or in a line section branching off from this line. Such an arrangement of the pressure sensor means is particularly advantageous because the ventilation or irrigation line is at all times flushed only with air and cannot therefore become blocked. This ensures at all times a precise determination, by the pressure sensor means, of the negative pressure applied in the wound space.

The valve normally receives the opening pulses via a communication link with the control means. It is also conceivable to provide the ventilation valve with an autonomous opening mechanism.

It is important in all the embodiments according to the invention that the pressure sensor means is in fluidic communication with the wound space at the time the negative pressure is determined. A fluidic communication with the wound space can also be established indirectly, for example via the drainage line or via the drainage container.

According to the invention, the wound therapy device comprises a means for delivering an instillation fluid from the instillation container into the wound space. In a straightforward embodiment of the invention, the means for delivering an instillation fluid from the instillation container into the wound space can be, for example, an infusion stand. In this embodiment, the instillation container is mounted such that the instillation fluid can flow into the wound by gravity.

Moreover, it is also possible that the instillation fluid is sucked into the wound space solely by the suction force of the negative pressure applied to the wound.

According to a preferred embodiment of the invention, the means for delivering an instillation fluid from the instillation container into the wound space is a pump unit, wherein the pump unit is connected to the instillation container in a fluid-conducting manner. When a predefined lower negative-pressure threshold value is reached in the wound space, the pump unit can be switched off, such that the further delivery of the instillation fluid is interrupted. It is switched off in particular in response to a signal output by the control means when a predefined lower negative-pressure threshold value is reached in the wound space.

The means for delivering an instillation fluid from the instillation container into the wound space is preferably a second pump unit (fluid delivery pump) provided in addition to the first pump unit. The fluid delivery pump has a communication link with the control means. When a predefined lower negative-pressure threshold value is reached in the wound space, the control means outputs, via the communication link, a signal to switch off the second pump unit, such that the further delivery of the instillation fluid is interrupted.

According to an alternative embodiment of the invention, the instillation therapy device comprises a valve or shut-off valve, which can close the fluid-conducting connection between the instillation container and the negative-pressure dressing. A communication link is present between the valve and the control means, such that the valve can be closed by the control means when a lower negative-pressure threshold value is reached. The further delivery of the instillation fluid is interrupted by the closure of the valve.

Provision can also be made that the device simultaneously comprises a valve and a second pump unit. In such cases, the closure of the valve and the simultaneous switching-off of the second pump unit can better ensure that no further instillation fluid is introduced into the wound when a predefined lower negative-pressure threshold value is reached in the wound space.

Even at a negative pressure of less than 30 mmHg (pressure difference from the ambient pressure), the negative pressure in the wound approximates to the ambient pressure. It is thus ensured that an optimal quantity of instillation fluid has been introduced into the wound. According to a preferred embodiment of the invention, the lower threshold value for the negative pressure is between 0 mmHg and 30 mmHg, preferably between 5 mmHg and 25 mmHg, and still more preferably between 10 mmHg and 20 mmHg.

According to a preferred embodiment of the invention, the instillation therapy device is portable. Portable means that the shape and weight of the instillation therapy device are such that it can be carried around by a patient. Moreover, a portable instillation therapy device must have a suitable and autonomous power supply, for example a rechargeable battery. A portable instillation therapy device promotes the mobility of the patient.

It also proves advantageous if the wound therapy device has a temperature-regulating element for regulating the temperature of the instillation fluid. The temperature-regulating element comprises a temperature sensor. The temperature-regulating element is preferably arranged in the interior of the instillation container or on the outside thereof. Alternatively to this, the temperature-regulating element could also be arranged on the instillation line and change only the temperature of the instillation fluid introduced into the wound. This can be advantageous, for example, when using instillation solutions that contain a heat-sensitive medicament. Regulating the temperature of the instillation fluid, for example at 30° C. to 37° C., can promote the wound-healing process. In particular, an instillation therapy that is greatly improved by comparison with the prior art can be carried out by administering a temperature-controlled instillation fluid in a quantity that is adapted exactly to the volume of the wound space.

The instillation fluid preferably comprises a liquid. Liquids are particularly suitable for detaching necrotic tissue, cleaning the wound and thinning the wound liquid. In this way, liquids contribute to the efficient debridement of the wound. According to another preferred embodiment, the instillation fluid comprises at least one buffer substance. By applying liquids that comprise buffer substances, the pH value of the wound can be influenced, and a wound environment that is particularly conducive to the wound-healing process can thus be established. When applying an instillation liquid that comprises one or more buffer substances, it is particularly important to suitably meter the delivered volume of liquid, since otherwise it may not be possible to achieve the desired effect of influencing the pH value of the wound.

Optionally, the liquid can contain active substances in dissolved form or as colloids. The active substances can be bound to carrier substances. Examples of suitable active substances with properties that promote the wound-healing process are antiseptics, antibiotics, growth factors, clotting factors, or nutrients that support tissue healing.

The liquid preferably comprises water or an electrolyte solution, in particular Ringer's solution. An electrolyte solution, in particular an isotonic electrolyte solution, can advantageously be used as instillation solution, since it has a salt concentration similar to the body fluids and is therefore osmotically inactive. An osmotically inactive instillation liquid is particularly gentle on tissue and supports the wound-healing process. According to a particularly preferred embodiment, the irrigation liquid comprises Ringer's solution. The advantageous effect of Ringer's solution in the negative-pressure therapy of wounds is particularly marked in what is called the cleaning phase of the wound-healing process. Ringer's solution is understood as a synthetic solution which is approximately isosmotic to blood and which contains sodium chloride, potassium chloride and calcium chloride. Ringer's solution can be produced, for example, by dissolving 8.6 g of sodium chloride, 0.30 g of potassium chloride and 0.33 g of calcium chloride (dihydrate) in a liter of distilled water.

According to a further concept of the invention, it can prove advantageous if an instillation liquid with hypertonic properties is used in the instillation therapy. An instillation liquid with hypertonic properties has a higher salt concentration by comparison with the salt concentration of the body fluids. An instillation liquid with hypertonic properties is osmotically active and supports the release of tissue liquid into the wound space. The use of an instillation liquid with hypertonic properties can be particularly suitable for the therapy of wounds with large amounts of necrotic tissue.

According to an independent concept of the invention, it proves advantageous if the instillation fluid comprises a gas.

It also proves advantageous in this connection if the gas is chosen from the gases air, oxygen-enriched air, or nitrogen. An advantage of air is that the air is available everywhere and can be used immediately after suitable sterilization. Oxygen-enriched air is advantageous because it can stimulate growth of the tissue in the wound space. Oxygen-enriched air is understood here as air that has a greater content of oxygen than the ambient air, i.e. an oxygen content of more than 21%. The use of nitrogen is advantageous, since nitrogen has a very high degree of compatibility with skin and tissue. According to a further concept of the invention, it is proposed that the gas comprises one or more atomized active substances in aerosol form. Provision can also be made that the gas comprises odor-absorbing substances.

### Use of the Device According to the Invention

A polyurethane foam that has been cut to the size of the wound to be treated is placed onto the wound. The polyurethane foam is in direct contact with the wound surface. The polyurethane foam and the area immediately surrounding the wound are covered with an adhesive, fluid-tight cover film, such that a wound space is formed between cover film and wound base. The film adheres to the intact skin surrounding the patient's wound and closes the wound space in a gas-tight manner. A negative-pressure attachment means (port) is secured on the film, by means of which fluidic connections are established between the wound space and the instillation line, between the wound space and the drainage line, and between the wound space and, if present, the irrigation line.

By starting up the negative-pressure pump (first pump unit), a negative pressure is generated in the wound space, i.e. in the space formed between cover film and wound base. The negative pressure present in the wound space is detected and continuously monitored by a pressure sensor means, which communicates with the control means. As soon as the pressure sensor means registers a negative pressure above a predefined upper threshold value, the first pump unit is switched off. The upper threshold value can, if appropriate, be manually predefined by a user before the wound therapy, for example by way of an input unit. A suitable input unit is, for example, a keyboard or a touchscreen. The upper threshold value could, for example, be a negative pressure of between 75 mmHg and 150 mmHg. The upper threshold value preferably lies in the range of between 100 mmHg and 130 mmHg and is in particular 100 mmHg, 125 mmHg or 130 mmHg.

After the inactivation of the negative-pressure pump, the means for delivering the instillation fluid is activated, such that instillation fluid can pass into the wound space. The means for delivering the instillation fluid is preferably a second pump unit (fluid delivery pump), which is controlled by the control means.

The negative pressure in the wound space decreases as a result of the delivery of instillation fluid into the wound. A decrease in the air pressure is understood here as meaning that the negative pressure approaches the ambient air pressure. The decreasing negative pressure is registered by the pressure sensor means. As soon as the negative pressure reaches a lower threshold value, the second pump unit is switched off by the control means. Like the upper threshold value, the lower threshold value can be manually predefined by a user, optionally before the wound therapy, for example by way of an input unit. The lower threshold value chosen can, for example, be a negative pressure of between 0 mmHg and 35 mmHg, in particular a negative pressure of 0 mmHg, 5 mmHg, 10 mmHg, 15 mmHg, 20 mmHg, 25 mmHg or 30 mmHg.

The instillation fluid then remains in the wound for a predefined time period, for example 30 seconds to 10 minutes, and during this time can act on the wound base.

At the end of the predefined time period, the first pump unit can be switched on again, and the instillation fluid is aspirated together with the wound liquid via the drainage line, until the upper threshold value for the negative pressure in the wound space is once again reached. Irrigation and suction cycles of this kind can be repeated through to the end of the instillation therapy.

### BRIEF DESCRIPTION OF THE DRAWINGS

Embodiments of wound therapy devices according to the invention are explained in more detail below with reference to the drawings. However, the invention should not be understood as being limited to the embodiments set forth in the drawings or in the description of the drawing. Rather, the invention also covers combinations of the individual features of the alternative forms.

FIG. 1 shows a first embodiment of the wound therapy device according to the invention (schematic view). The wound therapy device shown comprises a first and a second pump unit.

FIG. 2 shows another embodiment of the instillation therapy device according to the invention (schematic view). The wound therapy device shown comprises a first pump unit.

FIG. 3 shows another embodiment of the wound therapy device according to the invention, which has a pressure meter arranged in the fluid path between the drainage container and the first pump unit (schematic view).

FIG. 4 shows another embodiment of the wound therapy device according to the invention, which has an additional pressure measurement line (schematic view).

### KEY TO THE FIGURES

1 drainage container
2 pressure sensor means
3 drainage line
4 first pump unit (negative-pressure pump)
5 control means
7 instillation container
8 instillation line
9 second pump unit (fluid delivery pump)
10 display
11 input unit
12 temperature-regulating element
13 temperature sensor
15 negative-pressure dressing
16 negative-pressure attachment piece (port)
17 cover film
20, 40, 50, 60 wound therapy device
21 irrigation line
22 irrigation valve
23 bacteria filter
41 irrigation or ventilation valve
16 pressure measurement line

### DETAILED DESCRIPTION OF THE DRAWINGS

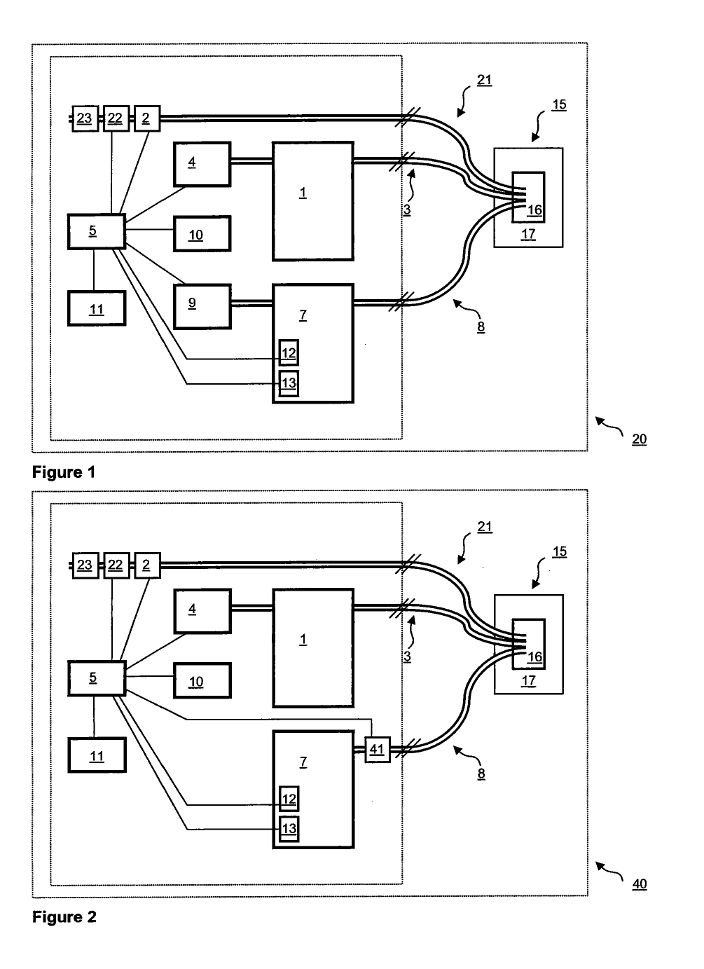

FIG. 1 shows a wound therapy device 20 according to the invention. The wound therapy device 20 for the treatment of wounds comprises a drainage container 1 for collecting liquid. The container 1 is made of a plastic and has a volume of ca. 800 ml. The wound therapy device further comprises a drainage line 3, which connects the first container 1 in a fluid-conducting manner to the negative-pressure dressing 15 and to the wound space. The drainage line 3 comprises a flexible plastic hose, for example of silicone, with an inner open cross-sectional area of 10 to 200 mm², for example. The negative-pressure dressing 15 comprises an open-cell polyurethane foam as wound pad (not shown) and a cover film 17, wherein the polyurethane foam below the cover film 17 is applied to the wound base. In the embodiment shown, the drainage line 3, the instillation line 8 and the irrigation line 21 are connected to the cover film 17 by means of a single port 16.

The irrigation line 21 is in fluidic communication with the pressure sensor means 2. In the embodiment shown in FIG. 1, the pressure sensor means 2 is arranged in the interior of the irrigation line 21. At its end area oriented away from the wound, the irrigation line 21 has the pressure sensor means 2, the irrigation valve 22 and the bacteria filter 23. The pressure measurement data determined on the pressure sensor means 2 are forwarded to the control means 5 via a communication link. The pressure sensor means 2 is arranged in the interior of the irrigation line 21 or, alternatively, in a line section (not shown) that branches off from the irrigation line 21, and it thus ensures monitoring of the negative pressure in the wound space as long as the irrigation valve 22 is closed.

The first pump unit (negative-pressure pump) 4 is connected to the first container 1 in a fluid-conducting manner and is controlled by the control means 5. The negative-pressure pump 4 comprises a diaphragm pump, which can be controlled directly via the control means 5. The control means 5 is an electronic component, preferably a programmable electronic component, in particular a programmable microprocessor. A suitable input unit 11, for example a keyboard or a touchpad, is preferably connected to the electronic component, in order to allow the user to input programs for negative-pressure therapy and instillation therapy. The control means 5 preferably has an indicator means in the form of a display 10. A communication link, for example in the form of a cable for serial data transfer, is present between the control means 5 and the pressure sensor means 2.

The wound therapy device comprises a second container (instillation container) 7 for making available an instillation fluid, and an instillation line 8 which connects the instillation container 7 to the wound space in a fluid-conducting manner. Like the first container 1, the instillation container 7 is a container made of plastic and having a volume of ca. 800 ml. Optionally, the instillation container 7 could also be designed as a flexible bag impermeable to liquid. Ringer's solution is preferably used as instillation fluid.

In the preferred embodiment of the invention shown in FIG. 1, the means for delivering an instillation fluid from the instillation container 7 into the wound space comprises a second pump unit (fluid delivery pump) 9, which is controlled by the control means 5. For this purpose, a communication link is provided between the fluid delivery pump 9 and the control means 5. The fluid delivery pump 9 is connected to the instillation container 7 in a fluid-conducting manner and effects the delivery of instillation fluid into the wound space. Like the first pump unit (negative-pressure pump) 4, the fluid delivery pump 9 can comprise a diaphragm pump.

A temperature-regulating element 12 with a temperature sensor 13 is optionally provided in the instillation container 7 and is able to change the temperature of the instillation fluid. The temperature sensor 13 is connected to the control means 5 by a cable, in particular by a cable for serial data transfer.

The embodiment shown in FIG. 1 is portable and is provided for mobile use. All the electrical and electronic components are powered by a rechargeable battery (not shown).

FIG. 2 shows an alternative embodiment of the wound therapy device 40 according to the invention. The wound therapy device 40 for the treatment of wounds differs from the device 20 shown in FIG. 1 in that a valve 41 is provided between the instillation container 7 and the instillation line 8. The valve 41 is provided in order to close the fluid-conducting instillation line 8 between the second container 7 and the negative-pressure dressing 15 when a predefined lower negative-pressure threshold value in the wound space is reached (for example 10 mmHg). In contrast to the wound therapy device 20, no fluid delivery pump is present. Instead, an alternative means is provided for delivering an instillation fluid from the container 7. In the simplest case, the instillation fluid is delivered to the wound space by the suction force of the negative pressure applied to the wound. According to the embodiment shown in FIG. 2, a communication link is present between the valve 41 and the control means 5.

FIG. 3 shows another alternative embodiment of the wound therapy device 50 according to the invention. The wound therapy device 50 differs from the device 20 shown in FIG. 1 in that the pressure sensor means 2 is arranged in the fluid path between drainage container 1 and first pump unit 4.

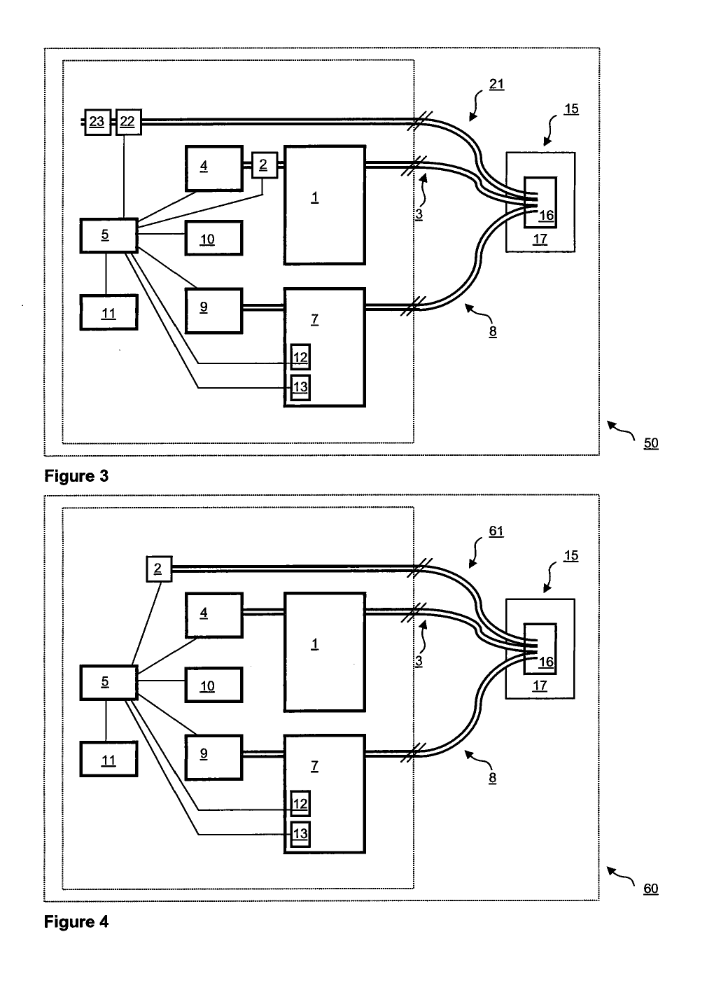

In the embodiment shown in FIG. 4, the wound therapy device 60 comprises an additional pressure measurement line 61 between wound dressing 15 and pressure sensor means 2. The additional pressure measurement line 61 constitutes a fluidic connection between wound space and pressure sensor means 2. Proximally, therefore, the additional pressure measurement line 61 ends on the negative-pressure dressing 15, wherein a fluidic connection to the wound space can be provided, for example, by means of a negative-pressure attachment piece. The pressure sensor means 2 is located at the distal end of the pressure measurement line 61 and is preferably arranged inside the housing of the therapy device. Since the pressure sensor means 2 is thus in fluid communication with the wound space via the pressure measurement line 61, the negative pressure in the wound space can be monitored by a pressure sensor means mounted on the therapy device or in the therapy device.

1. Wound therapy device (20, 40, 50, 60) for the treatment of wounds by means of negative pressure and fluid irrigation, comprising
i. a negative-pressure dressing (15) with a fluid-tight cover, such that a wound space can be formed between the cover and the wound base,
ii. a pressure sensor means (2), which can generate pressure measurement values, wherein the pressure sensor means (2) is in fluidic communication with the wound space,
iii. a drainage container (1) for collecting liquid,
iv. a drainage line (3) by means of which liquid can be conveyed from the wound space into the drainage container (1),
v. a first pump unit (4) for aspirating liquid from the wound space via the drainage line (3), wherein the first pump unit (4) is connected in a fluid-conducting manner to the drainage container (1),
vi. an instillation container (7) for making available an instillation fluid,
vii. an instillation line (8) by means of which instillation fluid can be delivered from the instillation container (7) into the wound space,
viii. a means for delivering an instillation fluid from the instillation container (7) into the wound space,
ix. a control means (5), wherein the control means (5) can receive pressure measurement values from the pressure sensor means (2),
characterized in that
x. when a predefined upper negative-pressure threshold value is reached in the wound space, the control means can output a signal to switch off the first pump unit (4), and
xi. when a predefined lower negative-pressure threshold value is reached in the wound space, the control means can output a signal to interrupt the delivery of the instillation fluid.

2. Wound therapy device (20, 40, 50, 60) according to claim 1, further comprising a means for determining the volume of instillation fluid that is introduced into the wound space.

3. Wound therapy device (20, 50, 60) according to claim 1, wherein the means for delivering an instillation fluid from the instillation container (7) into the wound space is a second pump unit (9), which is connected in a fluid-conducting manner to the instillation container (7).

4. Wound therapy device (40) according to claim 1, further comprising a valve (41) which can close the fluid-conducting connection between the instillation container (7) and the wound space, wherein the signal output by the control means to interrupt the delivery of the instillation fluid causes the closure of the valve (41).

5. Wound therapy device (20, 40, 50, 60) according to claim 1, wherein the pressure sensor means (2) is arranged in the wound space.

6. Wound therapy device (20, 40, 50, 60) according to claim 1, wherein the pressure sensor means (2) is in fluidic communication with the drainage line (3) and/or the drainage container (1).

7. Wound therapy device (20, 40, 50, 60) according to claim 1, wherein the drainage container (7) is arranged in the fluid path between the first pump unit (4) and the drainage line (3).

8. Wound therapy device (50) according to claim 1, wherein the pressure sensor means (2) is arranged in the fluid path between the first pump unit (4) and the drainage container (1).

9. Wound therapy device (60) according to claim 1, wherein the wound therapy device has a separate pressure measurement line (61), and wherein the pressure measurement line (61) is in fluidic communication with the negative-pressure dressing (15) and with the pressure sensor means (2).

10. Wound therapy device (20, 40, 50) according to claim 1, wherein the wound therapy device has a ventilation line or an irrigation line (21), and wherein the ventilation line or irrigation line (21) is in fluidic communication with the pressure sensor means (2).

11. Wound therapy device (20, 40, 50, 60) according to claim 1, wherein the lower negative-pressure threshold value in the wound space is between 0 mmHg and 30 mmHg, preferably between 5 mmHg and 25 mmHg, and still more preferably between 10 mmHg and 20 mmHg.

12. Wound therapy device (20, 40, 50, 60) according to claim 1, wherein the upper negative-pressure threshold value in the wound space is between 100 mmHg and 130 mmHg, in particular between 120 mmHg and 130 mmHg.

13. Wound therapy device (20, 40, 50, 60) according to claim 1, wherein the wound therapy device is stationary or portable.

14. Wound therapy device (20, 40, 50, 60) according to claim 1, wherein the wound therapy device has a temperature-regulating element (12) for regulating the temperature of the instillation fluid.

15. Wound therapy device (20, 40, 50, 60) according to claim 14, wherein the temperature-regulating element (12) comprises a through-flow heating element.

16. Wound therapy device (20, 40, 50, 60) according to claim 1, wherein the instillation fluid comprises a liquid.

17. Wound therapy device (20, 40, 50, 60) according to claim 16, wherein the liquid is chosen from water, electrolyte solution or Ringer's solution.

18. Wound therapy device (20, 40, 50, 60) according to claim 1, wherein the instillation fluid comprises a gas.

19. Wound therapy device (20, 40, 50, 60) according to claim 18, wherein the gas is chosen from the gases air, oxygen-enriched air, or nitrogen.

20. Method for metering a suitable volume of instillation fluid for the instillation therapy of wounds, comprising:
a. Making available a wound therapy device (20, 40, 50, 60) according to claim 1;
b. Applying a negative-pressure dressing (15) to the wound, wherein a wound space is formed;
c. Switching on the first pump unit (4) in order to generate a negative pressure in the wound space;
d. Monitoring the negative pressure in the wound space by means of the pressure sensor means (2);
e. Switching off the first pump unit (5) when an upper negative-pressure threshold value is reached in the wound space;
f. Delivering an instillation fluid from the instillation container (7) through the instillation line (8) into the wound space until a lower negative-pressure threshold value is reached.

21. Method for determining a suitable volume of instillation fluid for the instillation therapy of wounds, comprising:
a. Making available a wound therapy device (20, 40, 50, 60) according to claim 1;
b. Applying a negative-pressure dressing (15) to the wound, wherein a wound space is formed;
c. Switching on the first pump unit (4) in order to generate a negative pressure in the wound space;
d. Monitoring the negative pressure in the wound space by means of the pressure sensor means (2);
e. Switching off the first pump unit (5) when an upper negative-pressure threshold value is reached in the wound space;
f. Delivering an instillation fluid from the instillation container (7) through the instillation line (8) into the wound space until a lower negative-pressure threshold value is reached;
g. Determining the volume of the instillation fluid delivered in step f), and storing the determined volume;
